(19) United States
(12) Patent Application Publication
(10) Pub. No.: US 2023/0346731 A1
(43) Pub. Date: Nov. 2, 2023

(54) AMINO ACID COMPOSITIONS AND FUNCTIONAL FOOD AFFECTING TUMOR GROWTH AND USES THEREOF

(71) Applicant: Chengdu Shangyi Information Technology Co., Ltd., Chengdu (CN)

(72) Inventors: Xuelei Ma, Chengdu (CN); Chengyuan He, Chengdu (CN); Huimin He, Chengdu (CN); Jun Yin, Chengdu (CN); Zhiwen Long, Chengdu (CN)

(21) Appl. No.: 18/306,254

(22) Filed: Apr. 25, 2023

(30) Foreign Application Priority Data
Apr. 28, 2022 (CN) 202210460140.1

(51) Int. Cl. A61K 31/198; A23L 33/00; A23L 33/175; A23J 3/22; A61P 35/00

(52) U.S. Cl. CPC A61K 31/198; A23L 33/40; A23L 33/175; A23J 3/227; A61P 35/00

(57) ABSTRACT

The present disclosure provides an amino acid composition and functional food affecting tumor growth and the use thereof, which falls within the technical field of amino acids. The present disclosure is capable of effectively inhibiting the growth of cancer cells and tumor tissues by strictly limiting the content of serine and glycine and increasing alanine content through cancer cell culture and studies of the transcriptome and metabolic mechanism. Therefore, the functional protein or food developed by the present disclosure, with strictly limited serine and glycine content and increased alanine content, can be advantageous in exerting an anti-tumor effect.

### REFERENCE TO ELECTRONIC SEQUENCE LISTING

The application contains a Sequence Listing which has been submitted electronically in .XML format and is hereby incorporated by reference in its entirety. Said .XML file, created on Apr. 25, 2023, is named “Seqlist_XML.xml” and is 13,203 bytes in size.

### TECHNICAL FIELD

The present disclosure falls within the technical field of amino acids, and specifically relates to amino acid composition and functional food affecting tumor growth and use thereof.

### BACKGROUND

Cancer is one of the significant public health problems worldwide, accounting for the forefront global cause of death.

Cancer therapies are currently focused on surgical therapy, radiation therapy, chemotherapy, biological treatment, hormone therapy, etc., while diet, as the fundamental guarantee for the treatment of cancer patients, merely focuses on improving nutritional status; it per se has no apparent therapeutic effect. The metabolism of nutrients differs in tumor cells from normal cells. A tumor is an abnormal tissue formed due to changes in cellular genetic material, odd gene expression, and abnormal cell proliferation under various tumorigenic factors. Tumor cells can avoid growth inhibition and even utilize normal cells to create a tumor microenvironment (TME) conducive to tumor growth and immune destruction to ensure continuous proliferation and survival. Metabolism in tumor cells varies from that in normal ones, wherein the metabolism of amino acids plays a vital role in tumor cell proliferation. Amino acid metabolism in tumor cells consumes a large proportion of energy consumption, accounting for 33 to 58% of the total energy consumption of cancer cell proliferation, and the consumption of leucine and serine ranks at the forefront. Amino acids participate in protein biosynthesis during tumor cell proliferation and provide carbon and nitrogen sources for synthesizing ribonucleic acid (RNA) and deoxyribonucleic acid (DNA), allowing rapid growth of tumor cells. Serine and glycine are important sources of the one-carbon unit. Theoretically, we may inhibit the proliferation and growth of tumor cells by regulating the metabolism of tumor cells through the limitation of the amino acid level in food, i.e., limiting exogenous amino acids available to tumor cells. In fact, it is found by several animal studies that by limitation of certain non-essential amino acids, serum amino acid levels can be reduced, with tumor cell growth being inhibited and therapeutic effects of anti-tumor medicals being enhanced.

Currently, there is no food for tumor patients to limit/adjust amino acids to inhibit tumor growth commercially available. Diet is a nutritional guarantee for tumor patients, and it is thus necessary to develop food for tumor patients to limit/adjust amino acids as a dietary choice for tumor patients for tumor therapy.

### SUMMARY

In view of this, it is one object of the present disclosure to provide an amino acid composition that affects tumor growth, which is proved to be able to produce the effects that inhibit the production of tumor cells through transcriptional and metabolic pathway studies, while the amino acids included are non-essential ones for normal cells.

It is also an object of the present disclosure to provide a functional food for tumor patients, which can achieve obvious anti-tumor effect by adjusting the amino acid components and the content thereof.

The present disclosure provides an amino acid composition affecting tumor growth, comprising serine and glycine.

Preferably, the composition further comprises alanine.

The present disclosure provides use of the amino acid composition in the preparation of an anti-tumor product.

Preferably, serine and glycine in the amino acid composition are essential amino acids for the growth of cancer cells, and the content of serine and glycine is inhibited in the product to exert an anti-tumor effect.

Preferably, content of alanine in the amino acid composition of the product is increased for the purpose of anti-tumor effect.

The present disclosure provides a method for treating tumor patient, comprising administering to the tumor patient a therapeutically effective amount of the amino acid composition.

Preferably, serine and glycine in the amino acid composition are essential amino acids for the growth of cancer cells.

Preferably, content of alanine in the amino acid composition is increased for the purpose of anti-tumor effect.

The present disclosure provides a functional protein comprising the following amino acids in mass percentage: serine 0-0.00108%, glycine 0-0.0015%, alanine 4-20%, and the balance are amino acids required for normal cell growth.

Preferably, the amino acids required for normal cell growth comprise 10% to 94% essential amino acids by mass percentage. The essential amino acids include the following components in parts by weight: 16.1 to 26.5 parts of leucine, 11.5 to 20.1 parts of lysine, 11.04 to 15.65 parts of valine, 9.66 to 16.1 parts of phenylalanine, 4.6 to 11.5 parts of methionine, 8.74 to 13.8 parts of isoleucine, 8.7 to 12.7 parts of threonine and 2.2 to 6.5 parts of tryptophan.

The amino acids required for normal cell growth further comprise 0% to 86% non-essential amino acids and conditionally essential amino acids. The non-essential amino acids and conditionally essential amino acids comprise one or more amino acids selected from the group consisted of glutamic acid, aspartic acid, asparagine, glutamine, arginine, proline, tyrosine, cysteine, and histidine.

The present disclosure provides functional food comprising the functional protein and excipients.

Preferably, the functional food further comprises dietary nutrients.

The present disclosure provides use of the functional protein or functional food for improving the diet of tumor patients;

Preferably, the tumor comprises at least one of the following: colorectal cancer, breast cancer, lymphoma, pancreatic cancer, lung cancer, and liver cancer.

The present disclosure provides an amino acid composition affecting tumor growth comprising serine and glycine. In the present disclosure, cancer cells are used as a cultured control to respectively evaluate the effect on cell growth in the absence of one or two of 13 amino acids; a variety of cancer cells, such as colorectal cancer cells, non-Hodgkin's lymphoma cells, non-small cell lung cancer cells, small cell lung cancer cells, liver cancer cells, breast cancer cells, and pancreatic ductal adenocarcinoma cells and the like, are more sensitive in the absence of serine and glycine in the culture medium; and the growth of cancer cells recovers while replenished with serine and glycine. It is indicated that serine and glycine are essential amino acids for the growth of cancer cells. It may benefit from the absence of serine and glycine in the culture medium for inhibiting the growth of many cancer cells.

Further, the present disclosure discloses that limiting alanine content can also affect tumor growth. Through transcriptional and metabolic mechanism studies, there is a lipid metabolism disorder in tumor tissues lacking serine and glycine. Upon alanine content is increased, it further promotes the synthesis of cytotoxic doxSLs in tumor cells, thereby inhibiting the growth of cancer cells or tumor tissues.

The present disclosure provides a functional protein comprising the following amino acids in mass percentage: serine 0-0.00108%, glycine 0-0.0015%, alanine 4-20%, and the balance are amino acids required for normal cell growth. The cellular and animal level tests of the present disclosure demonstrate that increasing alanine levels and by severely limiting serine and glycine levels can effectively inhibit the growth of cancer cells or tumor tissue. Thus, the functional protein can serve as the sole dietary protein to provide a protein source for tumor patients.

Further, the functional protein of the present disclosure further limits the levels of essential amino acids, non-essential amino acids, and conditionally essential amino acids in order to ensure the normal growth of human cells and prevent the occurrence of malnutrition.

The present disclosure also provides functional food, which combines the above functional protein with carbohydrates, fats, dietary fiber, vitamins, minerals, and other nutrients required by the human body to obtain the formula food suitable for tumor patients and can be used as a full-day diet for tumor patients in special cases, such as the inconvenience of oral intake, tube feeding nutrition, etc. The population pre-experiments results showed that serine and glycine levels in the serum of tumor patients who only took functional food decreased initially and then increased. Serine and glycine levels after the intervention were still lower than before the intervention. The serum immunity levels of CD3⁺ and CD4⁺ increased while CD8⁺ decreased during the intervention.

The functional food also has the following properties:
1) Unlike the current medical food for tumor patients commercially available, which only focuses on improving the nutritional status of patients, the present disclosure adjusts the composition of amino acids by studying the regulation mechanism of transcription and metabolism of tumor cells after restricting amino acids, and microscopically and precisely optimizes nutritional therapy, so that the auxiliary effect of nutritional therapy on tumor diseases is not limited to improving the nutritional status of patients, but also providing a new idea and new method for the treatment of tumors.
2) Unlike the present disclosure based on in vitro cell culture or animal models, the study design of the present disclosure based on transcriptional and metabolic regulatory mechanisms is more convincing, more feasible and safer to extrapolate to humans. The population pre-experiments have also demonstrated that serum serine and glycine level will decrease with the restriction of dietary amino acid, and it is worthy to continue the research in tumor treatment;
3) Diet is the most fundamental and necessary requirement for tumor patients daily. The present disclosure provides another more suitable choice for the diet of tumor patients on a molecular level; which is economical and essential, and may enhance the effect of clinical therapy.

### BRIEF DESCRIPTION OF THE DRAWINGS

FIG. 1 is a transcriptome differential analysis volcano plot;

FIG. 2 shows the functional enrichment analysis result of differentially expressed genes;

FIG. 3 is a volcano plot of differential analysis of lipid metabolism;

FIG. 4 exhibits the trend of tumor volume change of HCT116 xenograft mice. *The tumor growth rate of Test Groups 3-5 is significantly less than Test Groups 1 and 2, P<0.05; # the tumor growth rate of Test Groups 1-5 is significantly less than Control Group (P<0.01);

FIG. 5 shows serum amino acids of xenografted HCT116 mice after 25 days of feeding, *indicates P<0.05, #indicates P<0.01;

FIG. 6 shows relative tumor amino acid levels in xenografted HCT116 mice after 25 days of feeding (Control growth level of 1). * indicates P<0.05, #indicates P<0.01;

FIG. 7 shows the change of serum albumin level in the tumor patient after taking the functional food;

FIG. 8 shows the change of plasma prealbumin level in the tumor patient after taking the functional food;

FIG. 9 shows the change of serum serine level in the tumor patient after taking the functional food.

FIG. 10 exhibits the change of serum glycine level in the tumor patient after taking the functional food;

FIG. 11 shows the change of serum alanine level in the tumor patient after taking the functional food;

FIG. 12 shows the change of serum CD3⁺, CD4⁺, and CD8⁺ levels in the tumor patient after taking the functional food.

### DETAILED DESCRIPTION

The present disclosure provides an amino acid composition affecting tumor growth, comprising serine and glycine.

In the present disclosure, it is found that when cancer cells are cultured in a medium in which specific amino acids are deleted, the cancer cell growth is inhibited upon the deletion of serine and glycine and is restored when replenished with serine and glycine, indicating that serine and glycine are essential amino acids for the growth of cancer cells. Increased levels of serine and glycine are beneficial in promoting tumor growth, and inhibition of serine and glycine contents can inhibit tumor growth, wherein cancer cells include cells of colorectal cancer, non-Hodgkin's lymphoma, non-small cell lung cancer, small cell lung cancer, liver cancer, breast cancer, pancreatic ductal adenocarcinoma and the like. Therefore, in the preparation of medicines or health products, or food from the viewpoint of anti-tumor, the anti-tumor purpose can be achieved by strictly limiting serine and glycine contents.

In the present disclosure, the amino acid composition further preferably comprises alanine. By analyzing the transcriptional and metabolic mechanisms of the inhibitory effects of serine and glycine on cancer cells, it was found that there was a lipid metabolism disorder in tumor tissues deficient in serine and glycine. Further analysis of lipid metabolites revealed that the level of doxSLs (deoxysphingolipids, doxSLs) was significantly increased, and alanine utilization of tumor cells deficient in serine and glycine was increased. Therefore, it was speculated that in the absence of serine, cancer cells would utilize alanine to synthesize cytotoxic doxSLs, which were shown to inhibit the growth of cancer cells. Therefore, as an “alternate” for serine, further increasing the alanine content can significantly inhibit the growth of cancer cells and tumor tissues based on strictly limiting the content of serine and glycine to achieve the purpose of anti-tumor.

The present disclosure provides the use of the amino acid composition in the preparation of an anti-tumor product, based on that the above amino acid composition content is capable of effectively modulating the growth characteristics of cancer cells or tumor tissues.

In the present disclosure, serine and glycine in the amino acid composition are essential amino acids for the growth of cancer cells; and the content of the serine and glycine in the product is inhibited to achieve the purpose of anti-tumor. The alanine content in the amino acid composition is increased in the product to achieve the purpose of anti-tumor. The present disclosure has no particular limitation on the product type, and it can be prepared into any form of anti-tumor product well known in the art, for example, a medicine, food, or health care product, etc.

The present disclosure provides a functional protein comprising the following amino acids in mass percentage: serine 0-0.00108%, glycine 0-0.0015%, alanine 4-20%, and the balance are amino acids required for normal cell growth.

In The present disclosure, in order to ensure the normal growth of human cells and prevent the occurrence of malnutrition, the required amino acids for normal cell growth preferably include all essential amino acids in percentage by weight of 10 to 96%. As the more closely the amino acid pattern of a protein is to the human body, the higher the biological value (BV) thereof is, i.e., the more easily it is digested and absorbed and utilized by the body. In order to increase the biological value of the amino acid composition according to the present disclosure, the essential amino acid should correspond to the following components in parts by weight: 16.1-26.5 parts of leucine, 11.5-20.1 parts of lysine, 11.04-15.65 parts of valine, 9.66-16.1 parts of phenylalanine, 4.6-11.5 parts of methionine, 8.74-13.8 parts of isoleucine, 8.7-12.7 parts of threonine and 2.2-6.5 parts of tryptophan. The amino acids required for normal cell growth preferably further comprise 0% to 86% non-essential amino acids and conditionally essential amino acids; the non-essential amino acids and conditionally essential amino acids include at least one of the following amino acids: glutamic acid, aspartic acid, asparagine, glutamine, arginine, proline, tyrosine, cysteine, and histidine.

The present disclosure provides a functional food comprising the functional protein and excipients.

In the present disclosure, the functional food is consumed as a protein supplement in combination with a protein-free diet as a full-day diet for special tumor patient. In the food, serine does not exceed 0.00018 wt %, and glycine does not exceed 0.00025 wt % of the total amount of the food; that is to say, the detectable value of serine and glycine is 0; alanine content accounts for more than 4 wt % of the total amino acids and more than 1.0 wt % of the total amount of food.

In the present disclosure, the forms of functional food preferably include tablets, solid powder, oral liquid, artificial meat, biscuit, bread, cake, beverage, gel, etc. Taking artificial meat as an example, the preparation method of the functional food is described. The excipients to the artificial meat are preferably binders, flavorings, and carriers. The carriers preferably include edible fungi, bamboo shoot coatings, and spices. The edible fungi preferably comprise at least one or more fungi selected from the group consisted of Pleurotus eryngii, Lentinus edodes, and Boletus. The flavorings include salt, pepper, meat flavor, glucose syrup, etc. The binders include carrageenan, starch, and the like. The artificial meat comprises the following components in parts by weight: 20-40 parts of carrier, 2-4 parts of carrageenan, 15-30 parts of sweet potato starch, 4-10 parts of glucose syrup, 12-18 parts of functional protein, 0.5-1.5 parts of salt, 0.7-2.1 parts of pepper and 0.01-0.1 parts of meat flavor essence; wherein the functional protein comprises 0.8-2.5 parts of L-leucine, 0.6-2 parts of L-lysine hydrochloride, 0.4-1.3 parts of L-valine, 0.3-1 parts of L-phenylalanine, 0.2-0.6 parts of L-methionine, 0.4-1.3 parts of L-isoleucine, 0.3-1 parts of L-threonine, 0.2-0.6 parts of L-tryptophan, 1-4.6 parts of L-glutamine, 0.6-2.1 parts of L-aspartic acid, 0.5-1.7 parts of L-arginine hydrochloride, 0.05-0.15 parts of L-alanine; 0.35-1.2 parts of L-proline, 0.16-0.55 parts of L-histidine, 0.3-1.0 parts of L-tyrosine, 0.2-0.65 parts of L-cystine. The amino acid composition in artificial meat accounts for about 3 wt % of the total food product. The method for the preparation of the functional food preferably includes the following steps: the carrier is made into a floe and mixed with flavorings, binders, and functional proteins and is subjected to enzymatic hydrolysis by glutamine transaminase to obtain artificial meat. The artificial meat can be eaten after frying, roasting or steaming, instead of protein-rich meat, soy products, etc. in the ordinary diet, and combined with a protein-free diet or a very low-protein diet, as a full-day diet for cancer patients.

In the present disclosure, the functional food is further preferably combined with carbohydrates, fats, dietary fiber, vitamins, minerals, and other dietary nutrients required by the human body to design formula food for special medical purposes suitable for specific tumor patients, as a full-day diet for tumor patients in special situations such as inconvenient to eat orally, tube-fed nutrition, etc. Foods for special medical purpose (FSMP) for particular medical use refers to the formula food specially prepared to meet the unique needs for nutrients or meals of people with restricted food intake, digestive and absorption disorders, metabolic disorders, or specific disease states (hereinafter referred to as “food for giving special medical treatment”). According to the above-mentioned principle of the disclosure, in combination with the relevant guideline recommendations and the requirements of “Reference Intake of Dietary Nutrients for Chinese Residents 2013 Edition” and “General Rules for Food Safety National Standard Formula for particular medical use” (GB29922-2013), a formula food for particular medical use suitable for specific tumor patients is designed to serve as the nutrient source for patients throughout the day. This “food for giving special medical treatment” contains carbohydrates, dietary fibers, proteins, fats, vitamins, and minerals that the human body requires daily. This “food for giving special medical treatment” comprises the following components in parts by weight: 170-533 parts of carbohydrate, all from maltodextrin; 23-80 parts of dietary fiber, all from pectin; 90-270 parts of plant-derived composite amino acid powder (amino acid composition) comprising 12-43 parts of L-glutamine, 10-25 parts of L-leucine, 10-22 parts of L-aspartic acid, 10-21 parts of L-lysine hydrochloride, 7-14 parts of L-arginine, 6-13 parts of L-valine, 5-11 parts of L-phenylalanine, 7-15 parts of L-alanine, 5-12 parts of L-proline, 6-13 parts of L-isoleucine, 5-12 parts of L-threonine, 2-6 parts of L-histidine, 5-10 parts of L-tyrosine, 3-6.5 parts of L-methionine, 3-6.5 parts of L-tryptophan, 3-7 parts of L-cystine; 160-520 parts of fat comprising 10-30 parts of octanoic acid, 6-21 parts of decanoic acid, 7-22 parts of palmitic acid, 3-10 parts of stearic acid, 27-81 parts of oleic acid, 30-80 parts of linoleic acid, 10-25 parts of α-linolenic acid; 1.5-4.5 parts of tropical fruit flavors; 0.2-0.6 parts of sucralose. The added amounts of vitamins and minerals as nutritional enhancers are as follows: 0.002-0.02 parts of vitamin A acetate, 0.00001-0.002 parts of cholecalciferol, 0.02-0.15 parts of dl-α-tocopherol acetate, 0.00015-0.001 parts of phytonadione, 0.003-0.12 parts of thiamine hydrochloride, 0.0041-0.02 parts of riboflavin, 0.008-0.03 parts of pyridoxine hydrochloride, 0.000005-0.0001 parts of cyanocobalamin, 0.02-1 parts of L-ascorbic acid, 0.0006-0.09 parts of folic acid, 0.01-0.1 parts of nicotinic acid; 0.01-0.09 parts of D-calcium pantothenate, 0.0001-0.009 parts of D-biotin, 10-40 parts of tricalcium carbonate, 3.3-12 parts of potassium chloride, 0.06-0.3 parts of dipotassium phosphate, 20-66 parts of sodium citrate, 0.8-3 parts of sodium chloride, 2-8 parts of magnesium sulfate, 0.0003-0.0015 parts of potassium iodate, 0.3-1.1 parts of iron pyrophosphate, 0.09-0.3 parts of zinc sulfate, 0.00024-0.0008 parts of sodium selenite, 0.005-0.02 parts of copper sulfate, 0.003-0.01 parts of manganese sulfate. The amino acid composition accounts for 17.7wt % of the total food in the food for giving special medical treatment. The carbohydrate, protein, and fat energy supply ratio in the “food for giving special medical treatment” is 30-50%:8-25%:30-50%. The method for preparing the “food for giving special medical treatment” preferably comprises the following steps:
1) Vitamin, edible essence, sucralose, and 50% of the total mass of maltodextrin are mixed to obtain Premix 1; wherein the total amount of vitamins, edible essence, and sucralose is weighed with maltodextrin at a mass ratio of 10:1;
2) minerals and remaining maltodextrin are mixed to obtain Premix 2;
3) Premix 1 and Premix 2 are mixed to obtain Premix 3;
4) Premix 3 is mixed with the remaining components to obtain a “food for giving special medical treatment” powder.

The “food for giving special medical treatment” as a full-day diet for specific tumor patients; the method of administration is preferable as follows: 45-80 g per time, 5-8 times per day, the mass ratio of powder and water is 1:4-8, brewed with warm water, can be taken orally and tube feeding can be performed (nasogastric/intestinal tube, gastric/intestinal fistula for tumor patients).

The present disclosure provides use of the functional protein or functional food for improving the diet of tumor patients.

In the present disclosure, the tumor preferably comprises at least one of the following: colorectal cancer, breast cancer, lymphoma, pancreatic cancer, lung cancer, and liver cancer.

The present disclosure provides an amino acid composition and functional food affecting tumor growth, and the use thereof, which are described in detail with reference to the following examples, but they should not be construed as limiting the scope of the present disclosure.

Sources of food for giving special medical treatments are shown in Table 1.

TABLE 1
Description of raw material sources

| | | Cat No/ |
|---|---|---|
| Raw material | Vendor | Product Code |
| Maltodextrin | Cargill Bio-Chemical Co., Ltd. | / |
| Plant-derived | INNOBIO Corporation Limited | 61B07-16B |
| complex amino | | |
| acid powder | | |
| Pectin | Cargill Bio-Chemical Co., Ltd. | AYS 400 YGM |
| Vegetable oil | INNOBIO Corporation Limited | 40506-110 |
| microcapsule | | |
| powder | | |
| Tropical fruit | Hasegawa Flavours (Suzhou) Co., | / |
| essence | Ltd. | |
| Sucralose | Anhui Jinhe Industry Co. Ltd. | / |
| Complex | DSM Vitimins (Shanghai) Limited | VR24470231, |
| minerals | | VR24471231 |
| Multivitamins | DSM Vitimins (Shanghai) Limited | VR24469231 |

### EXAMPLE 1

### Selection of Amino Acids by Cell Culture, Lacking Which May Have a Significant Inhibitory Effect on Cancer Cell Growth

In this test, various cancer cell lines were cultured in mediums lacking different non-essential amino acids to determine the amino acids without which the cancer cell growth is most significantly limited. The medium of Control Group is RPMI 1640 complete medium containing 10% fetal bovine serum and 1% double antibody (penicillin and streptomycin), etc. (vendor: Procell, Cat No: PM150110B), and 20 types of amino acids required for cell growth. The culture medium for the Test Group was the culture medium in which non-essential amino acids to be deficient are separated from it. Except for the missing amino acid, the other components are the same as the Control Group. The culture medium lacking non-essential amino acid was added with an equal volume of EBSS. Cancer cell line selection and amino acid deficiency in the culture medium of Test Groups are shown in Table 2 below.

TABLE 2
Selected cancer cell lines for test

| Cell line | Sources |
|---|---|
| HCT116 | Colorectal cancer |
| HL-60 | Acute promyelocytic leukemia |
| Jurkat | Acute T cell leukemia |
| U-937 | Non-Hodgkin's lymphoma |
| K-562 | Chronic myeloid leukemia |
| NCI-H1437 | Stage I, non-small cell lung cancer |
| NCI-H69 | Small cell lung cancer |
| HepG2 | Hepatocellular carcinoma |
| MDA-MB-231 | Breast cancer (adenocarcinoma) |
| HCC70 | Breast cancer (primary ductal carcinoma) |
| PC-3 | Adenocarcinoma of prostate |
| PATU-8902 | Pancreatic ductal adenocarcinoma |

TABLE 3
The amino acid deficiency in the culture medium of Test Groups

| Medium | Amino acid deficiency |
|---|---|
| -Ala | Alanine |
| -Arg | Arginine |
| -Asp | Aspartic acid |
| -Asn | Asparagine |
| -Cys | Cystine |
| -Gln | Glutamine |
| -Glu | Glutamic acid |
| -Ser | Serine |
| -Pro | Proline |
| -Gly | Glycine |
| -Tyr | Tyrosine |
| -SG | Serine and Glycine |

*As serine and glycine are interconvertible, a medium lacking Serine + Glycine was included in the test.

Cancer cell lines were cultured in different mediums; 3 days later, the growth of cancer cells was measured with CellTiter-Glo reagent (vendor: Promega, Cat No: G7570) to obtain the relative growth of cancer cells in the culture medium of Test Groups as shown in formula I.

Relative Growth=Reading (Test Groups)/Reading (Control Group) Formula I

The results are shown in Table 4. The missing amino acid with the same concentration of the complete medium was added to the growth-restricted cancer cell line. After 3-day incubation, a significant recovery of cancer cell growth was observed, indicating that the deletion of the amino acid caused the cell growth inhibition.

TABLE 4
Relative growth of cancer cells

| Cell line | -Ala | -Arg | -Asp | -Asn | -Cys | -Glu | -Gln | -Ser | -Pro | -Gly | -Tyr | -SG |
|---|---|---|---|---|---|---|---|---|---|---|---|---|
| HCT116 | 1.11 | 1.33 | 1.05 | 1.21 | 0.05 | 1.00 | 0.68 | 1.03 | 1.21 | 1.04 | 0.68 | 0.001 |
| HL-60 | 1.01 | 0.42 | 0.89 | 0.95 | 0.12 | 0.99 | 0.12 | 0.50 | 0.58 | 1.11 | 0.24 | 0.19 |
| Jurkat | 0.98 | 0.48 | 0.99 | 1.12 | 0.02 | 1.31 | 0.35 | 0.95 | 1.12 | 1.06 | 0.34 | 0.28 |
| U-937 | 0.94 | 0.28 | 0.9 | 0.96 | 0.21 | 1.11 | 0.20 | 0.83 | 0.99 | 0.79 | 0.44 | 0.002 |
| K-562 | 0.97 | 0.52 | 1.21 | 1.09 | 0.34 | 1.09 | 0.28 | 0.89 | 1.21 | 0.93 | 0.52 | 0.40 |
| NCI-H1437 | 1.01 | 0.55 | 0.89 | 0.90 | 0.18 | 0.34 | 0.36 | 0.54 | 1.09 | 0.89 | 0.63 | 0.002 |
| NCI-H69 | 0.97 | 0.30 | 0.83 | 0.81 | 0.10 | 0.95 | 0.26 | 0.28 | 1.01 | 0.84 | 0.16 | 0.07 |
| HepG2 | 0.88 | 0.35 | 0.98 | 1.05 | 06 | 0.96 | 0.7 | 0.62 | 0.89 | 0.76 | 0.61 | 0.001 |
| MDA-MB-231 | 0.91 | 0.65 | 1.10 | 1.04 | 0.001 | 1.16 | 0.38 | 0.53 | 1.12 | 0.71 | 0.48 | 0.003 |
| HCC70 | 0.92 | 0.68 | 1.02 | 1.11 | 0.005 | 1.03 | 0.69 | 1.11 | 1.17 | 0.97 | 0.56 | 0.02 |
| PC-3 | 1.09 | 0.6 | 1.22 | 1.20 | 0.04 | 1.19 | 0.53 | 1.09 | 1.26 | 1.09 | 0.66 | 0.22 |
| PATU-8902 | 1.03 | 0.72 | 1.05 | 1.33 | 0.55 | 1.13 | 0.71 | 0.87 | 1.08 | 0.79 | 0.78 | 0.003 |

*Cancer cells' lower relative growth ratio indicates a higher growth inhibition.

It is found that certain cancer cells were significantly inhibited in the medium lacking cysteine (−Cys), serine, and glycine (−SG). In −Cys medium, colorectal cancer cells, acute T-cell leukemia cells, liver cancer cells, breast cancer cells, and prostate cancer cells were significantly inhibited; In −SG medium, colorectal cancer cells, non-Hodgkin's lymphoma cells, non-small cell lung cancer cells, small cell lung cancer cells, liver cancer cells, breast cancer cells, pancreatic ductal adenocarcinoma cells are most sensitive to amino acid deficiency. Various cancer cells were well inhibited in the culture medium without serine and glycine.

### EXAMPLE 2

### Studies on Transcriptional and Metabolic Mechanisms of the Inhibition of Cancer Cells Due to Serine and Glycine Limitation

In this study, HCT116 cancer cell line was cultured in serine and glycine completely limited medium (−SG group) and complete medium (Con group), respectively. RNA-seq technique was used to detect the expression of genes in the Test Groups and Control Group. Based on DESeq algorithm, the corrected p value<0.05 and absolute value of logFC>1 was determined as the cut-off to screen differentially expressed genes and functional enrichment analysis was performed for the differentially expressed genes. Some of the differentially expressed genes were verified by q-PCR test. The primers were synthesized by Sangon Shanghai, with the specific sequences as shown in Table 5.

TABLE 5
Sequence primers information for
detection of various genes

| | Upstream primer | Downstream |
|---|---|---|
| Gene | sequence | primer sequence |
| PSAT1 | GGGTAGGTCCCGTCTACTCC | CCAAAGCCAATTCCATTCAC |
| | (SEQ ID NO: 1) | (SEQ ID NO: 2) |
| PSPH | CCACTCAGAGCTGAGGAAGC | TGAGCTTTGAAGCAACATGC |
| | (SEQ ID NO: 3) | (SEQ ID NO: 4) |
| SCD | CCCAGCTGTCAAAGAGAAGG | CAAGAAAGTGGCAACGAACA |
| | (SEQ ID NO: 5) | (SEQ ID NO: 6) |
| ASCT1 | CCTCACCATTGCCATTATCC | GCTTCCACTTTCACCTCAGC |
| | (SEQ ID NO: 7) | (SEQ ID NO: 8) |
| ASCT2 | GTCCTCACTCTGGCCATCAT | CTCCGTACGGTCCACGTAAT |
| | (SEQ ID NO: 9) | (SEQ ID NO: 10) |
| SPT | GTCCTCACTCTGGCCATCAT | ACGGTCCACGTAATTTTGGA |
| | (SEQ ID NO: 11) | (SEQ ID NO: 12) |
| 18S | AGTCCCTGCCCTTTGTACACA | CGATCCGAGGGCCTCACTA |
| rRNA | (SEQ ID NO: 13) | (SEQ ID NO: 14) |

Samples of 12, 24, and 48 hours post-cell transfection were collected and added with 0.5 ml MZ lysis solution. Total RNA was extracted according to the instructions of miRcute miRNA extraction and isolation kit, and measured the concentration; 1 ug RNA was reverse transcribed via iScript Reverse Transcription Supermix (Bio-Rad Laboratories) according to the instructions to obtain cRNA, and the expression level of the target gene was detected according to the instructions of SYBR GreenER Qpcr SuperMix Universal (Bio-Rad) kit, wherein each 10 μl reaction system comprises: 2 μl of diluted cRNA, 5 μl of SYBR Green Supermix and 1 μl of upstream and downstream primers. The reaction system of internal reference 18S rRNA was prepared by the same method above and then put into CFX Connect Real-time System (Bio-Rad CFX Manager v3.1) to perform the 3-stage reaction procedure: 3 min at 95° C., 40 cycles of 10 seconds at 95° C. and 30 sec at 60° C.

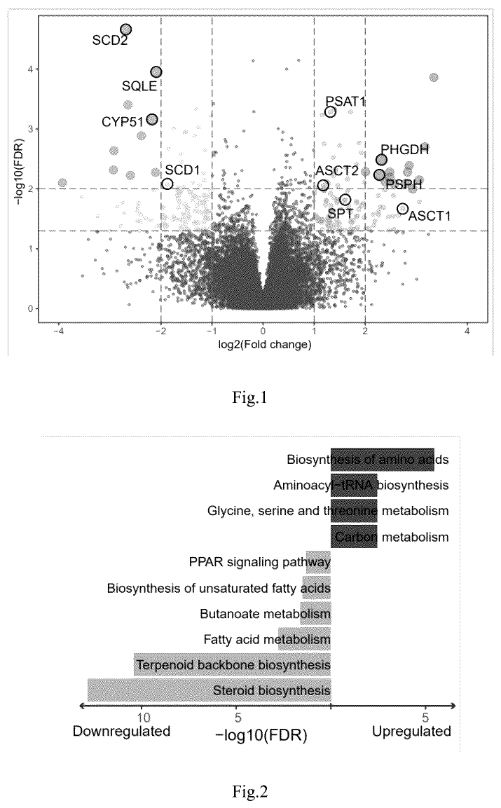

The rats were divided into IN Group (Test Group), NC Group (transfection non-sense oligonucleotide Group) and blank. As a result, in the tumor tissues of —SG Group (FIG. 1 and FIG. 2):
\textcircled{1} Up-regulation of genes PSAT1 and PSPH related to de novo serine synthesis reveals the decrease of intracellular serine level;
\textcircled{2} Down-regulation of the expression of crucial enzyme SCD1 in fatty acid metabolism and enrichment of dysregulated gene function shows the imbalance of lipid metabolism in tumor tissues with deficiency of serine and glycine;
\textcircled{3} Up-regulation of the expression of neutral amino acid transporters (ASCT1 and ASCT2) suggests increased uptake of neutral amino acids; by detection of amino acid contents in medium, it is found that alanine content significantly decreased in the medium of −SG Group, i.e., tumor cells deficient in serine and glycine have increased utilization of alanine;
\textcircled{4} Serine palmitoyltransferase (SPT) expression is upregulated.

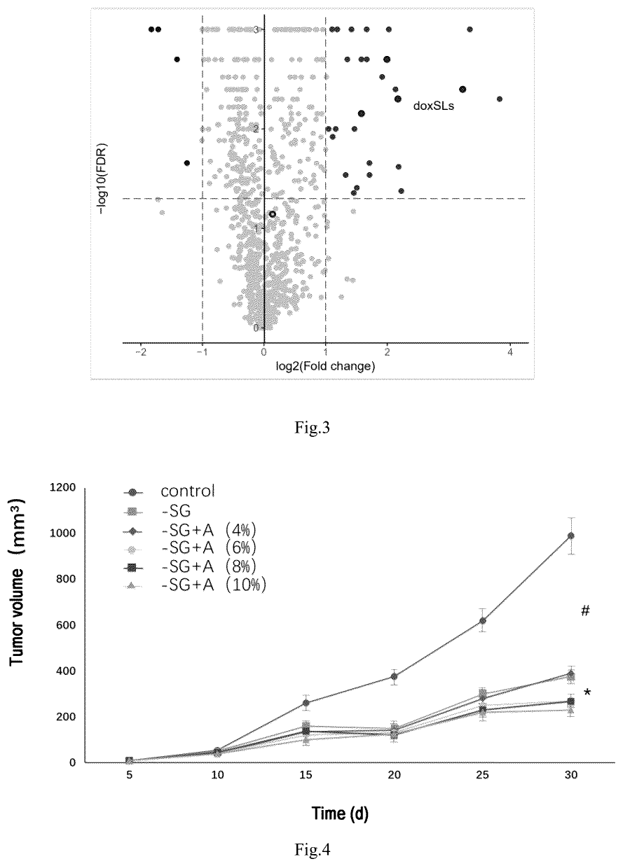

The results of the transcriptomic analysis show a lipid metabolism disorder in tumor tissues lacking serine and glycine. Furthermore, we performed gas chromatography-mass spectrometry (GC-MS) analysis on lipid metabolites and calculated the abundance of each metabolite using the INCA algorithm. After difference analysis, we found that doxSL (deoxysphingolipids) levels were significantly elevated in the tumor tissues of the —SG group (FIG. 3).

From the results of the above tests, it can be speculated that alanine utilization is increased in the medium lacking serine and glycine. It affects the lipid metabolic pathway and produce doxSLs having an antitumor effect, thereby inhibiting cancer cell growth.

### EXAMPLE 3

### Exploration of the Anti-Cancer Effect of Different Doses of Alanine Combined With Serine and Glycine Deficiency

Study in mouse:

In the above test, it was found that alanine utilization in cancer cells was increased in the medium lacking serine and glycine to produce cytotoxic substances. Then, in the test, the mice injected with the human colorectal cancer cell line (HCT116) were divided into Control and Test groups. The Control group was fed the feed containing all amino acid formulas (Control). The feed of Test Group 1 had an alanine content (wt %) identical to that of Control group without serine and glycine (—SG). The feed of Test groups 2-5 contained an alanine content identical to that of Control group without serine and glycine, with increased alanine content accounting for 4 wt %, 6 wt %, 8 wt %, and 10 wt % of total amino acids, respectively (mouse feed vendor: Biopike, M19053001). See Table 6 for component information. The total amount of amino acids in Control group and Test groups account for 16.84-18.46 wt % of the total amount of feed. Before dietary intervention, the mice in the Test groups and Control group were fed with regular feed, and the dietary intervention began after feeding until the tumor tissue size was detectable, and the growth of tumor cells in the experimental mice was observed.

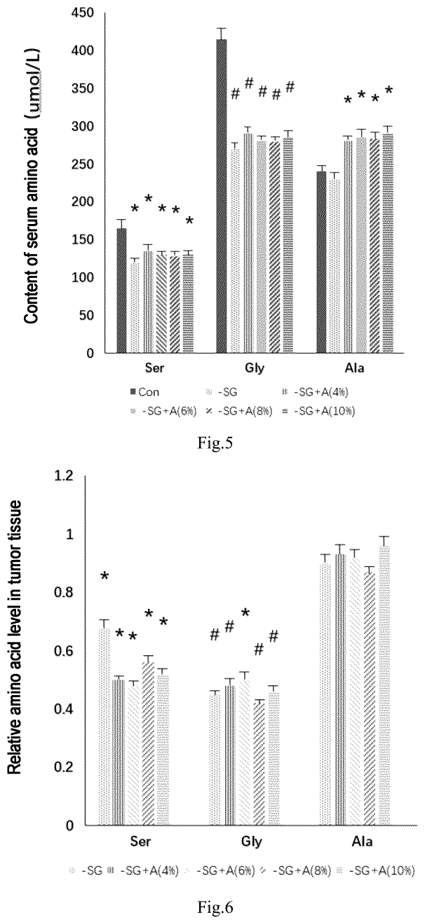

It was found that: the growth rate and tumor volume of mouse tumor tissues of Test Groups were significantly lower than Control group (FIG. 4). When alanine content accounts for ≥6 wt % of total amino acids, the tumor growth of mice was significantly inhibited, and the growth rate was apparently lower than Control group, −SG group and −SG+A (4%) group. After dietary intervention for 25 days, serum serine and glycine levels significantly decreased in Test group. Serum alanine levels increased dramatically in Test groups 3-5 (FIG. 5). The serine and glycine levels in the tumor tissues of mice in Test Groups significantly decreased. The alanine level was not entirely different from that of the Control (FIG. 6).

TABLE 6
Comparison of main components of feed for mice in each group

| | Content (g/100 g) | | | | | |
|---|---|---|---|---|---|---|
| | Control | Test | Test | Test | Test | Test |
| Ingredient | Group | Group 1 | Group 2 | Group 3 | Group 4 | Group 5 |
| Carbohydrate | 66.78 | 68.4 | 68.24 | 67.88 | 67.52 | 67.12 |
| Fat | 5.1 | 5.1 | 5.1 | 5.1 | 5.1 | 5.1 |
| Cellulose | 5.1 | 5.1 | 5.1 | 5.1 | 5.1 | 5.1 |
| Protein | 18.46 | 16.84 | 17 | 17.36 | 17.72 | 18.12 |
| L-Arginine | 0.61 | 0.61 | 0.61 | 0.61 | 0.61 | 0.61 |
| L-His-HCl-H₂O | 0.47 | 0.47 | 0.47 | 0.47 | 0.47 | 0.47 |
| L-Isoleucine | 0.77 | 0.77 | 0.77 | 0.77 | 0.77 | 0.77 |
| L-Leucine | 1.61 | 1.61 | 1.61 | 1.61 | 1.61 | 1.61 |
| L-Lysine-HCl | 1.34 | 1.34 | 1.34 | 1.34 | 1.34 | 1.34 |
| L-Methionine | 0.82 | 0.82 | 0.82 | 0.82 | 0.82 | 0.82 |
| L-Phenylalanine | 0.85 | 0.85 | 0.85 | 0.85 | 0.85 | 0.85 |
| L-Threonine | 0.73 | 0.73 | 0.73 | 0.73 | 0.73 | 0.73 |
| L-Tryptophan | 0.21 | 0.21 | 0.21 | 0.21 | 0.21 | 0.21 |
| L-Valine | 0.95 | 0.95 | 0.95 | 0.95 | 0.95 | 0.95 |
| L-Alanine | 0.55 | 0.52 | 0.68 | 1.04 | 1.4 | 1.8 |
| L-Asparagine-H₂O | 0.68 | 0.68 | 0.68 | 0.68 | 0.68 | 0.68 |
| L-Aspartic acid | 0.55 | 0.55 | 0.55 | 0.55 | 0.55 | 0.55 |
| L-Cystine | 0.12 | 0.12 | 0.12 | 0.12 | 0.12 | 0.12 |
| L-Glutamic acid | 2.2 | 2.2 | 2.2 | 2.2 | 2.2 | 2.2 |
| L-Glutamine | 1.67 | 1.67 | 1.67 | 1.67 | 1.67 | 1.67 |
| L-Glycine | 0.41 | 0 | 0 | 0 | 0 | 0 |
| L-Proline | 1.8 | 1.8 | 1.8 | 1.8 | 1.8 | 1.8 |
| L-Serine | 1.21 | 0 | 0 | 0 | 0 | 0 |
| L-Tyrosine | 0.94 | 0.94 | 0.94 | 0.94 | 0.94 | 0.94 |
| Mineral Mixture, | 3.56 | 3.56 | 3.56 | 3.56 | 3.56 | 3.56 |
| S10001 | | | | | | |
| Vitamin Mixture, | 1 | 1 | 1 | 1 | 1 | 1 |
| V14901 | | | | | | |
| Protein energy | 18.63% | 16.96% | 17.13% | 17.49% | 17.85% | 18.25% |
| supply ratio | | | | | | |
| Carbohydrate | 67.24% | 68.91% | 68.75% | 68.38% | 68.02% | 67.62% |
| energy supply ratio | | | | | | |
| Fat energy supply | 11.56% | 11.56% | 11.56% | 11.56% | 11.56% | 11.56% |
| ratio | | | | | | |

In light of the result, we can conclude that: dietary restriction of serine and glycine is effective in inhibiting tumor growth in HCT116 mice. When the proportion of alanine in the total amount of amino acids is ≥6wt %, it can limit the growth of tumor tissue more effectively than simply limiting serine and glycine.

### EXAMPLE 4

A functional full-nutrition powder and the preparation method thereof

An amino acid composition is used as the protein source in the nutritional powder, wherein the type and content of the amino acids in the composition are specifically listed in Table 7, wherein serine and glycine are not contained, and the content of alanine is increased. Other nutrients in the nutritional powder are designed according to the recommendations of “Reference Intake of Dietary Nutrients for Chinese Residents (2013 Edition)”; see Table 8 for details.

TABLE 7
Amino acid content in amino acid
compound powder

| | Percentage |
|---|---|
| Name | (wt %) |
| L-Glutamine | 18.44 |
| L-Leucine | 10.67 |
| L-Aspartic acid | 9.40 |
| L-Lysine hydrochloride | 8.69 |
| L-Arginine hydrochloride | 7.28 |
| L-Valine | 5.34 |
| L-Phenylalanine | 4.46 |
| L-Alanine | 6.31 |
| L-Proline | 4.85 |
| L-Isoleucine | 5.34 |
| L-Threonine | 4.51 |
| L-Histidine | 2.33 |
| L-Tyrosine | 4.08 |
| L-Methionine | 2.72 |
| L-Tryptophan | 2.68 |
| L-Cystine | 2.91 |
| Serine | 0 |
| Glycine | 0 |

TABLE 8
Ingredients of full-nutritional powder

| | | Content per | Content per |
|---|---|---|---|
| | | 100 g | 100 kJ of |
| Item | Unit | powder | powder |
| Energy | kJ | 1793.18 | 100.00 |
| Proteins | g | 17.71 | 0.99 |
| Fat | g | 17.37 | 0.97 |
| Carbohydrate | g | 47.66 | 2.66 |
| Dietary fiber | g | 4.62 | 0.26 |
| Na | mg | 500.00 | 27.88 |
| Vitamin A | μg RE | 413.32 | 23.05 |
| Vitamin B1 | mg | 0.68 | 0.04 |
| Vitamin B2 | mg | 0.88 | 0.05 |
| Vitamin B6 | mg | 2.00 | 0.11 |
| Vitamin B12 | μg | 1.12 | 0.06 |
| Vitamin E | mg α-TE | 182.07 | 10.15 |
| Vitamin C | mg | 199.47 | 11.12 |
| Vitamin D | μg | 4.44 | 0.25 |
| Nicotinic acid | mg | 2.24 | 0.12 |
| Folic acid | μg | 131.12 | 7.31 |
| Zn | mg | 4.44 | 0.25 |
| Ca | mg | 444.44 | 24.79 |
| P | mg | 222.24 | 12.39 |
| Mg | mg | 141.16 | 7.87 |
| Fe | mg | 4.44 | 0.25 |
| K | mg | 586.68 | 32.72 |
| Cu | μg | 444.44 | 24.79 |
| Mn | ug | 222.24 | 12.39 |
| I | μg | 44.44 | 2.48 |
| Cl | mg | 658.16 | 36.70 |
| Se | μg | 22.24 | 1.24 |
| Vitamin K1 | μg | 33.32 | 1.86 |
| Pantothenic acid | mg | 2.24 | 0.12 |
| Biotin | μg | 22.24 | 1.24 |

### EXAMPLE 5

### Population Pre-Experiments

A 3-month pre-experiments was conducted in 10 hospitalized solid tumor patients with normal gastrointestinal function (see Table 9 for patient information). After ethical review and informed consent of the patients, the nutritional powder prepared in Example 4 was administered to the subject throughout the day. The subject's daily dose was 25-35 kcal/kg.d. The nutritional powder was added with a proper amount of warm water and stirred well before use, and then used without additional food. Subjects' body weight, serum albumin, prealbumin, CD3⁺, CD4⁺, CD8⁺, serum serine, glycine, and alanine were collected regularly and entrusted to the clinical laboratory of the hospital for completion.

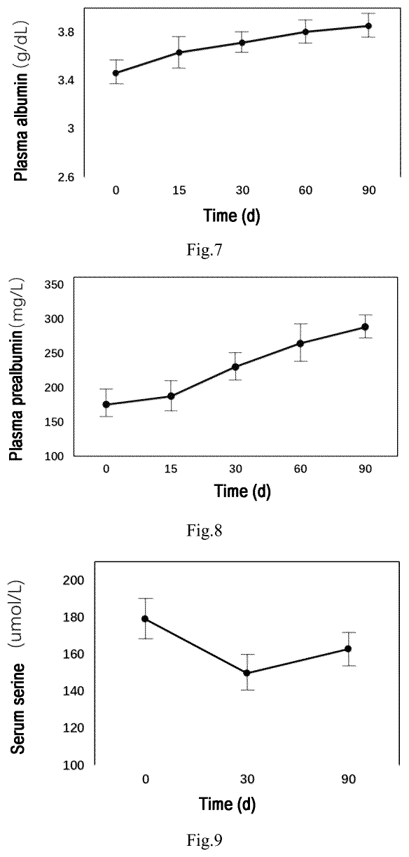

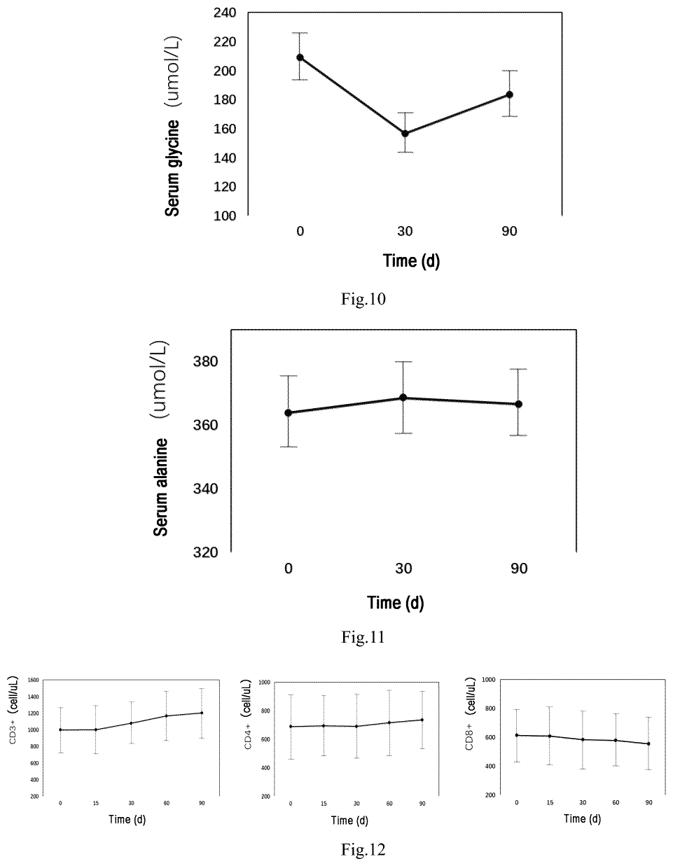

It was found that: the energy intake range of the subject throughout the day is 1500-1929 kcal, with protein: 62-80 g and fat: 61-78 g. The patient's serum albumin and prealbumin levels tended to increase (FIG. 7 and FIG. 8); serum serine and glycine levels decreased significantly after dietary intervention for one month and increased after intervention for three months but were still lower than that before intervention. The total serine level in serum is 179.0±13.2 μmol/L (day 0), 149.8±10.4 μmol/L (day 30), and 162.8±9.8 μmol/L (day 90); the decrease was 16.3% (day 30), and 9.1% (day 90); change of glycine level: 209.1±13.3 μmol/L (day 0), 156.8±11.9 μmol/L (day 30), and 183.4±13.2 ρmol/L (day 90); the decrease was 28.4% (day 30) and 12.3% (day 90). There was no significant change in alanine level before and after the study: 363.8±13.2 μmol/L (day 0), 368.6±13.6 μmol/L (day 30), 366.6±12.5 μmol/L (day 90) (FIG. 9, FIG. 10 and FIG. 11); immunological indicators CD3⁺ and CD4⁺ showed an upward trend, and CD8⁺ showed a downward trend (FIG. 12); all subjects had no significant weight loss during the intervention, and 3 of them had more than 5% weight gain. Nausea occurred in 2 patients, and vomiting occurred in 1 patient. There was no significant difference in the incidence of nausea and vomiting compared with that before the study (P>0.05). After symptomatic treatment, the patients recovered. After 20 days of feeding, the acceptance of nutritional powder decreased significantly and increased after changing the taste.

TABLE 9
Basic information of patients with solid tumor

| | | | Age |
|---|---|---|---|
| | Tumor type | Stage | (yrs) |
| Patient 1 | Colorectal cancer | IIIa | 55 |
| Patient 2 | Colorectal cancer | IIIa | 48 |
| Patient 3 | Colorectal cancer | IVa | 62 |
| Patient 4 | Colorectal cancer | IIIb | 46 |
| Patient 5 | Esophageal | IIIa | 56 |
| | carcinoma | | |
| Patient 6 | Esophageal | IIIb | 54 |
| | carcinoma | | |
| Patient 7 | Esophageal | IVb | 50 |
| | carcinoma | | |
| Patient 8 | Breast cancer | IIIb | 45 |
| Patient 9 | Breast cancer | IIIa | 58 |
| Patient 10 | Breast cancer | IIIa | 51 |

### EXAMPLE 6

### Formula Food (Powder) for Particular Medical Use Suitable for Tumor Patients

The raw materials of “food for giving special medical treatment” is comprised of the following: carbohydrate 355 g, all from maltodextrin; dietary fiber 51 g, all from pectin; 177 parts of a plant-derived complex amino acid powder (an amino acid composition) comprising 33 g of L-glutamine, 19 g of L-leucine, 17 g of L-aspartic acid, 16 g of L-lysine hydrochloride, 11 g of L-arginine, 10 g of L-valine, 8 g of L-phenylalanine, 11 g of L-alanine, 9 g of L-proline, 10 g of L-isoleucine, 8 g of L-threonine, 4 g of L-histidine, 7 g of L-tyrosine, 5 g of L-methionine, 5 g of L-tryptophan and 5 g of L-cystine; fat 174 g comprising caprylic acid 21 g, capric acid 14 g, palmitic acid 14 g, stearic acid 6g, oleic acid 54 g, linoleic acid 52 g, α-linolenic acid 14 g; tropical fruit essence 1.8 g; sucralose 0.1 g. The added amounts of vitamins and minerals as nutritional enhancers are as follows: vitamin A acetate 0.004 g, cholecalciferol 0.00004 g, dl-α-tocopherol acetate 0.07 g, phytonadione 0.0003 g, thiamine hydrochloride 0.007 g, riboflavin 0.009 g, pyridoxine hydrochloride 0.02 g, cyanocobalamin 0.00001 g, L-ascorbic acid 0.044 g, folic acid 0.0013 g, nicotinic acid 0.0224 g; D-calcium pantothenate 0.0224 g, D-biotin 0.0002 g, tricalcium carbonate 24 g, potassium chloride 7.4 g, dipotassium phosphate 0.17 g, sodium citrate 44.1 g, sodium chloride 1.82 g, magnesium sulfate 5 g, potassium iodate 0.00075 g, iron pyrophosphate 0.71 g, zinc sulfate 0.20 g, sodium selenite 0.0005 g, copper sulfate 0.011 g, manganese sulfate 0.007 g.

This “food for giving special medical treatment” is prepared in accordance with the following steps:
1) Ingredient Weighing: the electronic balance shall be calibrated before use. Accurately weigh each material in strict accordance with the formula according to the process procedures. Accurately weigh the premix with double-check. Premix 1 comprises vitamins, edible essences, sucralose, and part of maltodextrin (weighed with vitamins, edible essences, and sucralose in a ratio of 10:1); Premix 2 comprises minerals, part of maltodextrin (Premix 2: Premix 1=1:1); and Premix 3 comprises the final premix after mixing Premix 1 and Premix 2.
2) Sieve: all materials are dispersed and mixed through a 60-mesh oscillating sieve to ensure that there is no foreign matter in the product.
3) 1st Premix: the raw materials of Premix 1 were placed into a three-dimensional mixer for premixing for 20 minutes and put into a bag for later use after mixing.
4) 2nd Premix: the materials of Premix 1 and Premix 2 were put into a three-dimensional mixer for premixing for 20 minutes to obtain the final Premix 3, which was placed into a bag for later use.
5) Total mixing: Premix 3 was transferred from the three-dimensional mixer to the conical mixer, and the remaining materials were added for complete mixing for 30 minutes to obtain the end product.

The carbohydrate, protein, and fat energy supply ratio in the “food for giving special medical treatment” is 30-50%: 8-25%: 30-50%.

The nutrients are as follows:

### Method of Use

\textcircled{1} This “food for giving special medical treatment” is used as a full-day diet for patients with specific tumors;
\textcircled{2} 45-80 g per time, 5-8 times per day, the mass ratio of powder to water is 1:4.5g, warm water for reconstitution, may be orally administered and used for tube feeding (nasogastric/intestinal tube, gastric/intestinal fistula tumor patients).

### EXAMPLE 7

### An “Artificial Meat” as a Dietary Protein Supplement and the Preparation Thereof

### The preparation Steps of Artificial Meat

1) A total of 30 g of dried Pleurotus eryngii and bamboo shoot skin were soaked with 150 parts of clean water for about 3 h, and the soaked and softened bamboo shoot skin and Pleurotus eryngii floc were salvaged and drained for later use;
2) Added into the drained bamboo shoot skin and Pleurotus eryngii floc were the following: Carrageenan 3 g, sweet potato starch 25 g, glucose syrup 7 g, amino acid composition 15 g (Vendor: INNOBIO Corporation Limited) containing L-leucine 1.7 g, L-lysine hydrochloride 1.3 g, L-valine 0.83 g, L-phenylalanine 0.7 g, L-methionine 0.42 g, L-isoleucine 0.83 g, L-threonine 0.7 g, L-tryptophan 0.4 g, L-glutamine 2.34 g, L-aspartic acid 1.47 g, L-arginine hydrochloride 1.1 g, L-alanine 0.1 g, L-proline 0.8 g, L-histidine 0.36 g, L-tyrosine 0.65 g, L-cystine 0.43 g, salt 1 g, pepper 1.4 g, and meat flavor 0.05 g;
3) 0.4 g of glutamine aminotransferase was added into 5 g of distilled water for dissolution and then introduced into the mixture in step 2); the mixture was stirred evenly, placed into a mold, and maintained in the water bath at 40° C. for 3 h, allowing the enzyme to react sufficiently;
4) The artificial meat was removed from the mold, steamed on medium heat for 30 minutes, and stored in refrigeration.

Each 100 g of the above food contains 3 g of protein, 0.15 mg of serine accounting for 0.00015 wt % in the total food weight, 0.23 mg/100 g of glycine accounting for 0.00023 wt % in the total amino acid weight (the theoretical values of both serine and glycine are lower than the detection limit), and 1114 mg of alanine accounting for 6.8 wt % in the total amino acid weight and 1.114 wt % in the total food weight.

### Method of Use

The artificial meat prepared above is eaten after frying and roasting, and replaces protein-rich meat and soy products and the like in the ordinary diet, and is combined with a protein-free diet or a very low-protein diet as a full-day meal for cancer patients.

The above is only a preferred embodiment of the present disclosure, it should be pointed out that, for those of ordinary skill in the art, without departing from the principle of the present disclosure, some improvements and modifications can also be made, and these improvements and modifications should be regarded as within the protection scope of the present disclosure.

1. An amino acid composition that affects tumor growth, characterized in that the amino acid composition comprises serine and glycine.

2. The amino acid composition according to claim 1, characterized in that the amino acid composition further comprises alanine.

3. A method for treating tumor patient, comprising administering to the tumor patient a therapeutically effective amount of the amino acid composition of claim 1.

4. The method according to claim 3, characterized in that serine and glycine in the amino acid composition are essential amino acids for the growth of cancer cells.

5. The method according to claim 3, characterized in that content of alanine in the amino acid composition is increased for the purpose of anti-tumor effect.

6. A functional protein, characterized in that the functional protein comprises the following amino acids in mass percentage: serine 0-0.00108%, glycine 0-0.0015%, alanine 4-20%, and the balance are amino acids required for normal cell growth.

7. The functional protein according to claim 6, characterized in that the amino acids required for normal cell growth comprise 10% to 96% essential amino acids by mass percentage; the essential amino acids include the following components in parts by weight: 16.1 to 26.5 parts of leucine, 11.5 to 20.1 parts of lysine, 11.04 to 15.65 parts of valine, 9.66 to 16.1 parts of phenylalanine, 4.6 to 11.5 parts of methionine, 8.74 to 13.8 parts of isoleucine, 8.7 to 12.7 parts of threonine, and 2.2 to 6.5 parts of tryptophan;
preferably, the amino acids required for normal cell growth further comprise 0% to 86% non-essential amino acids and conditionally essential amino acids; the non-essential amino acids and conditionally essential amino acids comprise one or more amino acids selected from the group consisted of glutamic acid, aspartic acid, asparagine, glutamine, arginine, proline, tyrosine, cysteine, and histidine.

8. A functional food, characterized in that the functional food comprises the functional protein of claim 6 and excipients.

9. The functional food according to claim 8, characterized in that the functional food further comprises dietary nutrients.


SEQUENCE LISTING (14 sequences)

SEQ ID NO: 1 - DNA, 20 bp, synthetic construct
  source 1..20: mol_type = other DNA
gggtaggtcccgtctactcc

SEQ ID NO: 2 - DNA, 20 bp, synthetic construct
  source 1..20: mol_type = other DNA
ccaaagccaattccattcac

SEQ ID NO: 3 - DNA, 20 bp, synthetic construct
  source 1..20: mol_type = other DNA
ccactcagagctgaggaagc

SEQ ID NO: 4 - DNA, 20 bp, synthetic construct
  source 1..20: mol_type = other DNA
tgagctttgaagcaacatgc

SEQ ID NO: 5 - DNA, 20 bp, synthetic construct
  source 1..20: mol_type = other DNA
cccagctgtcaaagagaagg

SEQ ID NO: 6 - DNA, 20 bp, synthetic construct
  source 1..20: mol_type = other DNA
caagaaagtggcaacgaaca

SEQ ID NO: 7 - DNA, 20 bp, synthetic construct
  source 1..20: mol_type = other DNA
cctcaccattgccattatcc

SEQ ID NO: 8 - DNA, 20 bp, synthetic construct
  source 1..20: mol_type = other DNA
gcttccactttcacctcagc

SEQ ID NO: 9 - DNA, 20 bp, synthetic construct
  source 1..20: mol_type = other DNA
gtcctcactctggccatcat

SEQ ID NO: 10 - DNA, 20 bp, synthetic construct
  source 1..20: mol_type = other DNA
ctccgtacggtccacgtaat

SEQ ID NO: 11 - DNA, 20 bp, synthetic construct
  source 1..20: mol_type = other DNA
gtcctcactctggccatcat

SEQ ID NO: 12 - DNA, 20 bp, synthetic construct
  source 1..20: mol_type = other DNA
acggtccacgtaattttgga

SEQ ID NO: 13 - DNA, 21 bp, synthetic construct
  source 1..21: mol_type = other DNA
agtccctgccctttgtacaca

SEQ ID NO: 14 - DNA, 19 bp, synthetic construct
  source 1..19: mol_type = other DNA
cgatccgagggcctcacta